(19) United States
(12) Patent Application Publication
(10) Pub. No.: US 2026/0173259 A1
(43) Pub. Date: Jun. 18, 2026

(54) FLEXIBLE CIRCUIT BOARD AND ULTRASONIC PROBE

(71) Applicant: WUHAN UNITED IMAGING HEALTHCARE CO., LTD., Wuhan (CN)

(72) Inventors: Xiong Xiao, Wuhan (CN); Yuan Zhou, Wuhan (CN); Changxing Ke, Wuhan (CN); Kang Si, Wuhan (CN)

(21) Appl. No.: 19/411,351

(22) Filed: Dec. 7, 2025

(30) Foreign Application Priority Data
Dec. 13, 2024 (CN) 202411846578.9

(51) Int. Cl. H05K 1/02; A61B 8/00

(52) U.S. Cl. CPC H05K 1/028; A61B 8/4461; A61B 8/4494

(57) ABSTRACT

The present application provides a flexible circuit board and an ultrasonic probe. The ultrasonic probe includes a main shaft, a transducer, a signal processing circuit and the flexible circuit board. The flexible circuit board includes: a winding portion configured to be wound around the main shaft of the ultrasonic probe; a transduction connecting portion including connecting units arranged along a first direction, the at least two connecting units being foldable along the first direction, one end of each connecting unit being connected to a first end of the winding portion, and the other end of each connecting unit being configured to be electrically connected to the transducer; and an adapter connecting portion, one end of the adapter connecting portion being connected to a second end of the winding portion, and the other end of the adapter connecting portion being configured to be electrically connected to the signal processing circuit.

### RELATED APPLICATION

This application claims the benefit under 35 U.S.C. § 119 (a) of the filing date of Chinese Patent Application No. 2024118465789, filed in the Chinese Patent Office on Dec. 13, 2024. The disclosure of the foregoing application is herein incorporated by reference in its entirety.

### TECHNICAL FIELD

The present application pertains to the field of medical instrument technologies, and particularly relates to a flexible circuit board and an ultrasonic probe.

### BACKGROUND

An ultrasonic probe is mainly configured for ultrasonic detection in a cavity, and in an inspection process, a motor and a linear array transducer are usually arranged in the ultrasonic probe, and the motor drives the linear array transducer to rotate by 360 degrees to collect a three-dimensional image. The linear array transducer includes a plurality of array elements distributed along an axial direction, a large number of signals need to be transmitted for the plurality of array elements, and therefore, a large number of signal transmission channels are required, resulting in a large width of a flexible circuit board.

### SUMMARY

The present application provides a flexible circuit board and an ultrasonic probe.

In an aspect, the present application provides a flexible circuit board for electrically connecting a transducer and a signal processing circuit in an ultrasonic probe, the flexible circuit board including:
a winding portion configured to be wound around a main shaft of the ultrasonic probe;
a transduction connecting portion including at least two connecting units arranged along a first direction, the at least two connecting units being capable of being folded along the first direction, one end of each connecting unit being connected to a first end of the winding portion, and the other end of each connecting unit being configured to be electrically connected to the transducer; and
an adapter connecting portion, one end of the adapter connecting portion being connected to a second end of the winding portion, and the other end of the adapter connecting portion being configured to be electrically connected to the signal processing circuit.

In some embodiments, each of the connecting units include a connecting section, and when the connecting units are folded, the connecting sections are arranged in a staggered manner to be electrically connected to the transducer respectively.

In some embodiments, the winding portion includes one winding unit, and the connecting units are respectively connected to the winding unit; or

the winding portion includes at least two winding units, a number of the winding units is the same as that of the connecting units, and the connecting units are connected to the winding units in a one-to-one correspondence manner; or

the winding portion includes at least two winding units, the number of the winding units is smaller than that of the connecting units, and each winding unit is connected to at least one connecting unit; or

the winding portion includes at least two winding units, the number of the winding units is larger than that of the connecting units, and each connecting unit is connected to at least one winding unit.

In some embodiments, a length extension direction of the winding portion is perpendicular to a length extension direction of the transduction connecting portion.

In some embodiments, the length extension direction of the winding portion is perpendicular to a length extension direction of the adapter connecting portion.

In some embodiments, the adapter connecting portion includes:
a fixed section, the fixed section being connected to the winding portion, and the fixed section being fixed relative to the signal processing circuit; and
an inserting section configured to be electrically connected to the signal processing circuit;
wherein the inserting section extends in a parallel or bent manner from the fixed section.

In some embodiments, after the at least two connecting units are folded, a width of the transduction connecting portion in the first direction is less than a sum of widths of all connecting units in the first direction.

In some embodiments, after the at least two connecting units are folded, a width of the transduction connecting portion in the first direction is equal to a width of one connecting unit in the first direction.

In some embodiments, each of the connecting units further includes a folding section, one end of the folding section is connected to the winding portion, and the other end of the folding section is connected to the connecting section; and
the connecting section includes a convex portion which extends outwards along the first direction relative to the folding section, and the convex portion is provided with an alignment hole for alignment with the transducer.
   In another aspect, the present application further provides an ultrasonic probe, including a main shaft, a transducer mounted at an output end of the main shaft, a signal processing circuit and a above flexible circuit board for electrically connecting the transducer and the signal processing circuit, the flexible circuit board comprising: a winding portion configured to be wound around a main shaft of the ultrasonic probe; a transduction connecting portion comprising at least two connecting units arranged along a first direction, the at least two connecting units being capable of being folded along the first direction, one end of each connecting unit being connected to a first end of the winding portion, and the other end of each connecting unit being configured to be electrically connected to the transducer; and an adapter connecting portion, one end of the adapter connecting portion being connected to a second end of the winding portion, and the other end of the adapter connecting portion being configured to be electrically connected to the signal processing circuit.

In some embodiments, the main shaft is provided with a slot, the slot at least penetrates a circumferential side wall of the main shaft, and the slot penetrates the output end of the main shaft; the folded connecting units are inserted into the slot and extend out of the output end of the main shaft to be electrically connected to the transducer.

In some embodiments, the transducer includes a plurality of array elements distributed sequentially along an axial direction, and each connecting section is electrically connected to one or more array elements.

In some embodiments, the ultrasonic probe further includes a connector, the transducer and the main shaft being connected through the connector, and each connecting unit being connected to the transducer via the interior of the connector.

In some embodiments, the ultrasonic probe further includes a protection member fixed relative to the signal processing circuit, the protection member being rotatably sleeved over the main shaft, the winding portion being wound between the main shaft and the protection member, and a circumferential side wall of the protection member being provided with a window for the adapter connecting portion to pass through.

In some embodiments, the adapter connecting portion is bonded to an outer wall of the protection member.

In some embodiments, the window corresponds to a central angle less than 90 degrees.

In some embodiments, the slot penetrates the circumferential side wall of the main shaft, and at least one insertion opening is formed on the circumferential side wall of the main shaft opposite to the slot, at a position corresponding to the winding portion.

In some embodiments, the ultrasonic probe further includes a limiting assembly, the limiting assembly being configured to limit a rotating stroke of the main shaft.

In some embodiments, the limiting assembly includes:
a blocking structure including a first blocking portion and a second blocking portion spaced apart along a circumferential direction;
a first limiting member fixedly connected to the main shaft, the first limiting member including an arc-shaped portion surrounding the main shaft, and the arc-shaped portion having a first limiting surface and a second limiting surface which are oppositely arranged along the circumferential direction; and
a second limiting member rotatably sleeved over the main shaft, the second limiting member including a first limiting portion and a second limiting portion, and the first limiting portion being movably arranged between the first limiting surface and the second limiting surface; the second limiting portion being movably arranged between the first blocking portion and the second blocking portion.

In some embodiments, the circumferential side wall of the main shaft is provided with a notch.

In some embodiments, a length of the winding portion is determined according to an outer diameter of the main shaft, an outer diameter of the protection member, and an inner diameter of the protection member.

### BRIEF DESCRIPTION OF THE DRAWINGS

In order to illustrate the technical solutions in the embodiments of the present application more clearly, the drawings required for describing the embodiments or the prior art will be described briefly. Apparently, the following described drawings are merely for some embodiments of the present application, and other drawings can be derived from these drawings by those of ordinary skill in the art without any creative effort.

FIG. 1 is a schematic structural diagram when connecting units in a flexible circuit board according to an embodiment of the present application are unfolded;

FIG. 2 is a schematic structural diagram after connecting units in the flexible circuit board according to the embodiment of the present application are folded;

FIG. 3 is a schematic structural diagram in which a winding portion in the flexible circuit board according to the embodiment of the present application is in a segmented structure;

FIG. 4 is a schematic structural diagram in which the winding portion in the flexible circuit board according to the embodiment of the present application is in an integral structure;

FIG. 5 is a schematic structural view of a bending connection of an adapter connecting portion in the flexible circuit board according to the embodiment of the present application;

FIG. 6 is a schematic exploded structural diagram of an ultrasonic probe according to an embodiment of the present application;

FIG. 7 is a schematic sectional structural diagram of the ultrasonic probe according to the embodiment of the present application;

FIG. 8 is a schematic diagram of an assembly structure of a flexible circuit board and a transducer in the ultrasonic probe according to the embodiment of the present application;

FIG. 9 is a schematic diagram of an assembly structure of the flexible circuit board, a main shaft and the transducer in the ultrasonic probe according to the embodiment of the present application;

FIG. 10 is a schematic exploded structural diagram of the main shaft, a protection member, a connector, the flexible circuit board and the transducer in the ultrasonic probe according to the embodiment of the present application;

FIG. 11 is a schematic enlarged structural diagram of part A in FIG. 10;

FIG. 12 is a schematic diagram of assembly of the main shaft, the protection member, the connector, the flexible circuit board and the transducer in the ultrasonic probe according to the embodiment of the present application;

FIG. 13 is a schematic diagram of a winding change of a winding portion between the main shaft and the protection member during rotation of the main shaft under a first winding condition;

FIG. 14 is a schematic diagram of a second extreme position of the winding portion between the main shaft and the protection member under the first winding condition;

FIG. 15 is a schematic diagram of a winding change of the winding portion between the main shaft and the protection member during rotation of the main shaft under a second winding condition;

FIG. 16 is a schematic structural diagram in which a notch is formed in the circumferential side wall of the main shaft;

FIG. 17 is a schematic structural diagram after a window of the protection member is enlarged;

FIG. 18 is a schematic exploded structural diagram of a limiting assembly in the ultrasonic probe according to the embodiment of the present application;

FIG. 19 is a schematic structural diagram of a first limiting member, a second limiting member and a stop ring in the ultrasonic probe according to the embodiment of the present application;

FIG. 20 is a schematic limiting diagram when the main shaft in the ultrasonic probe according to the embodiment of the present application rotates to a first state;

FIG. 21 is a schematic limiting diagram when the main shaft in the ultrasonic probe according to the embodiment of the present application rotates to a second state;

FIG. 22 is a schematic limiting diagram when the main shaft in the ultrasonic probe according to another embodiment of the present application rotates to the first state; and

FIG. 23 is a schematic limiting diagram when the main shaft in the ultrasonic probe according to the other embodiment of the present application rotates to the second state.

### DETAILED DESCRIPTION

The present application will be described in further detail below with reference to the accompanying drawings and embodiments in order to make the technical problem to be solved by the present application, technical solutions, and beneficial effects more clear. It should be understood that the specific embodiments described herein are only for explaining the present application, and not intended to limit the present application.

It should be noted that when an element is referred to as being “fixed on” or “provided at” another element, the element may be directly or indirectly located on the other element. When an element is referred to as being “connected to” another element, the element may be directly or indirectly connected to the other element.

It should be understood that, directions or positional relationships indicated by terms “length”, “width”, “upper”, “lower”, “front”, “rear”, “left”, “right”, “vertical”, “horizontal”, “top”, “bottom”, “inner”, “outer”, etc. are based on orientations or positional relationships shown in the accompanying drawings, and they are used only for describing the present application and for description simplicity, but do not indicate or imply that an indicated device or element must have a specific orientation or be constructed and operated in a specific orientation. Therefore, it cannot be understood as a limitation on the present application.

In addition, the terms such as “first” and “second” are used herein for purposes of description and are not intended to indicate or imply relative importance or significance or to imply the number of indicated technical features. Thus, the feature defined with “first” and “second” may include one or more of this feature explicitly or implicitly. In the description of the present application, “a plurality of” means two or more, unless otherwise specified.

As described above, the width of the flexible circuit board of the ultrasonic probe is large. However, it is difficult for the flexible circuit board with a large width to pass through a main shaft of the ultrasonic probe to be electrically connected to the transducer. The width of the flexible printed circuit refers to the lateral dimension in the flexible printed circuit's plane that is perpendicular to the axial direction of the array element distribution of the linear array transducer and perpendicular to the flexible printed circuit's own thickness, serving to accommodate parallel signal channels.

For example, in the related art, in order to achieve the purpose that the flexible circuit board passes through the main shaft to be electrically connected to the transducer, the main shaft is made into a hollow structure, one end of the flexible circuit board passes out from a center of the main shaft to be connected to the transducer, the other end of the flexible circuit board passes out from the center of the main shaft, and a winding coil is formed at a tail end of the main shaft and connected to a signal processing circuit. In some embodiments, the signal processing circuit includes an adapter board. As a part of the signal processing circuit, the adapter board will be directly described in the following description in place of the signal processing circuit, and electrical connection of a flexible wiring board to an adapter board will be described as an example. It can be easily understood that, in some embodiments, the signal processing circuit may not include the adapter board, and the flexible circuit board can be electrically connected to the signal processing circuit directly. Since the main shaft is hollow, the main shaft cannot be directly connected to the motor, primary gear transmission is required to be designed, and a reverse transmission gap is possibly generated; in addition, the main shaft is hollow and occupies a large space, which affects other structural designs of the ultrasonic probe.

Based on this, the present application provides a flexible circuit board and an ultrasonic probe, so as to solve the technical problem that the flexible circuit board is difficult to assemble due to the large width.

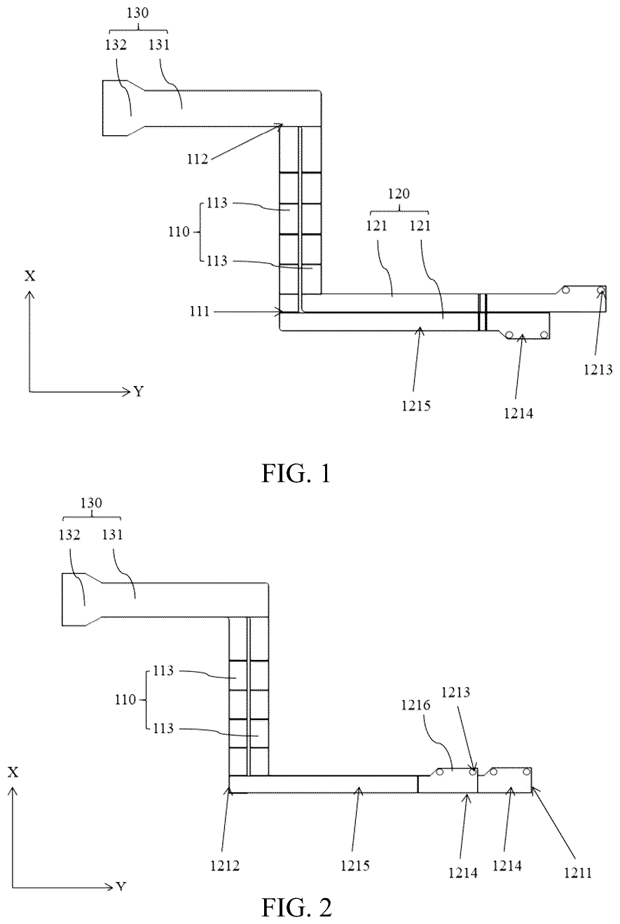

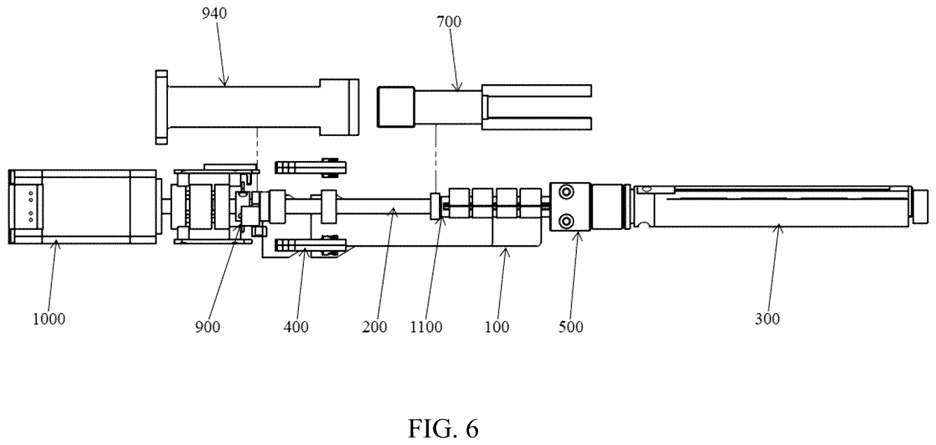

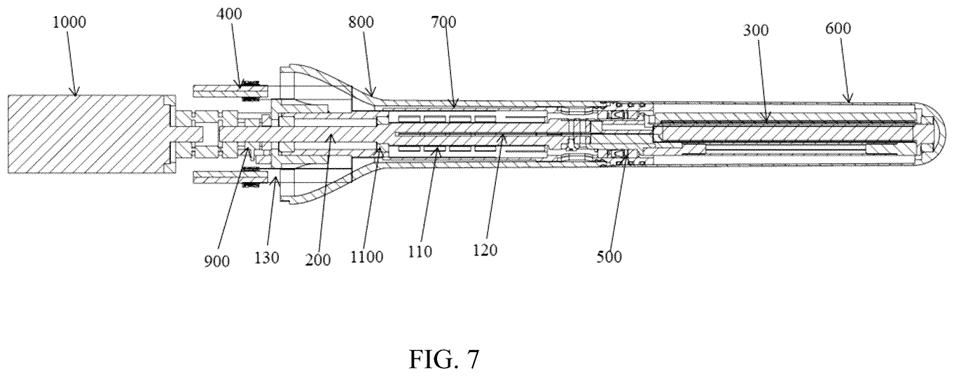

Referring to FIG. 1 to FIG. 3, FIG. 6 and FIG. 7 together, a flexible circuit board 100 according to an embodiment of the present application is described in detail. The flexible circuit board 100 is configured to electrically connect a transducer 300 and an adapter board 400 in an ultrasonic probe.

The flexible circuit board 100 includes a winding portion 110, a transduction connecting portion 120 and an adapter connecting portion 130; the winding portion 110 is configured to be wound around a main shaft 200 of the ultrasonic probe; the transduction connecting portion 120 includes at least two connecting units 121 arranged along a first direction X, the at least two connecting units 121 being foldable in the first direction X, one end of each connecting unit 121 being connected to a first end 111 of the winding portion 110, and the other end of each connecting unit 121 being configured to be electrically connected to the transducer 300; one end of the adapter connecting portion 130 is connected to a second end 112 of the winding portion 110, and the other end of the adapter connecting portion 130 is configured to be electrically connected to the adapter board 400.

The winding portion 110, transduction connecting portion 120, and the adapter connecting portion 130 are all parts of the flexible circuit board 100, and each part includes a base material layer and a metal layer, and can conduct electricity and conduct signals.

In the present application, the main shaft 200 of the ultrasonic probe Provides mechanical support and positioning, transmits motor torque to drive the transducer 300 for stable rotation, enabling 3D imaging. The transducer 300 converts electrical signals to ultrasonic waves (transmission) and echoes to electrical signals (reception), supplying raw data for 3D image reconstruction. The adapter board 400 facilitates signal transfer, interface adaptation and impedance matching, bridging the flexible circuit board 100 with the backend signal processing circuit.

Through the winding portion 110, the flexible circuit board 100 interfaces with the integrated signal channel group of the adapter board 400 (or the signal processing circuit), and the plurality of connection units 121 of the transducer connection part 120 interface with the plurality of array elements of the transducer 300, forming a “one input and multiple outputs” signal transmission mode.

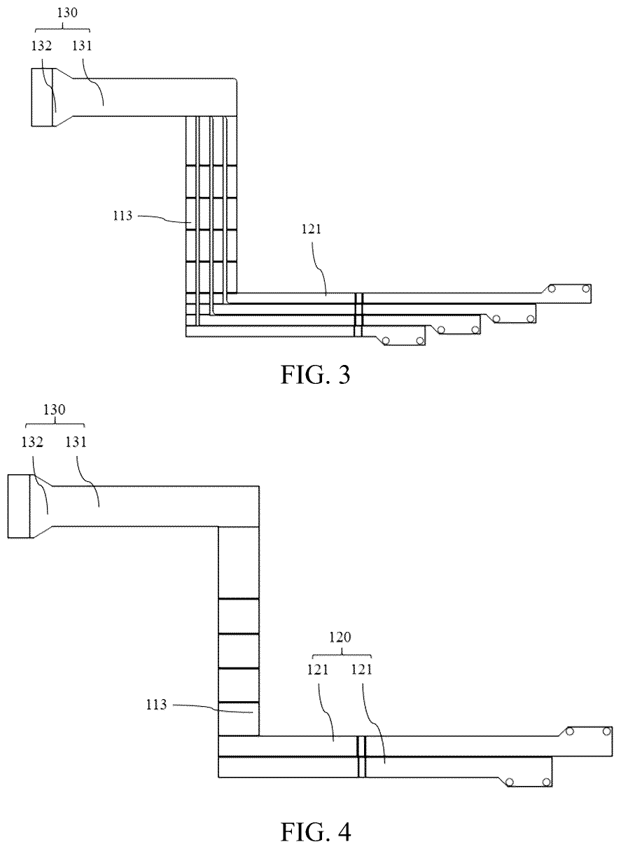

Referring to FIG. 1 and FIG. 3, two, three, four or more connecting units 121 may be provided.

In addition, it should be noted that the at least two connecting units 121 are foldable in the first direction X, which means that the at least two connecting units 121 are foldable towards the first direction X, and a direction of a folding axis may be a second direction Y perpendicular to the first direction X. The first direction X may be a length extension direction of the winding portion 110 extending from the first end 111 to the second end 112, and the connecting units 121 are distributed along the length extension direction of the winding portion 110 and are sequentially connected to the winding portion 110. Folding is performed from the connecting unit 121 farthest from the second end 112 towards the adjacent connecting unit 121 close to the second end 112 until the connecting units 121 are folded to the connecting unit 121 closest to the second end 112, that is, the connecting units 121 are overlapped with each other at the connecting unit 121 closest to the second end 112. FIG. 2 is a schematic diagram after the connecting units 121 are folded. It may be readily appreciated that the transduction connecting portion 120 in the present application has an unfolded state and a folded state. In the unfolded state, the connecting units 121 are not overlapped with each other, and in the folded state, the connecting units 121 are overlapped with each other at the connecting unit 121 closest to the second end 112.

The widths of the respective connection units 121 in the first direction X may be the same, and in this case, in the folded state, a width of the transduction connecting portion 120 in the first direction X is reduced to a width of one connecting unit 121 in the first direction X, thereby reducing the width of the transduction connecting portion 120 in the first direction X to at least half of the original width. Optionally, the widths of the respective connection units 121 in the X direction may be different, and in this case, in the folded state, the width of the transduction connecting portion 120 in the first direction X is reduced to a width of the connection unit 121 having the largest width in the first direction X. In other words, the width of the transduction connecting portion 120 in the first direction X is at least less than the sum of the widths of all the connecting units 121 in the first direction X. Therefore, the main shaft 200 does not need to be configured into a hollow structure, the connecting units 121 can be assembled by only forming a narrow gap in the main shaft 200, and in this case, a diameter of the main shaft 200 can be designed to be smaller. Certainly, in other embodiments, the length extension direction of the winding portion 110 may form an angle relative to the first direction X, which is not limited herein.

In the flexible circuit board 100 according to the embodiment of the application, the transduction connecting portion 120 is configured as the at least two connecting units 121, the at least two connecting units 121 are foldable in the first direction X, that is, the transduction connecting portion 120 is foldable in the first direction X, one end of each connecting unit 121 is connected to the first end 111 of the winding portion 110, and the other end of each connecting unit 121 is configured to be electrically connected to the transducer, so that the electrical connection of the adapter board 400 with the transducer can be realized, and meanwhile, the connecting units 121 can be folded in the first direction X during assembly, the total width of the transduction connecting portion 120 in the first direction X is equal to the width of one connecting unit 121, the width of the transduction connecting portion 120 in the first direction X is greatly reduced, and therefore, the transduction connecting portion 120 occupies a small space during assembly and can easily pass through the main shaft 200 to be electrically connected to the transducer 300. In addition, the arrangement of the winding portion 110 can prevent the flexible circuit board 100 from being pulled when the main shaft 200 and the transducer 300 rotate, and the risk that the flexible circuit board 100 is torn off is reduced.

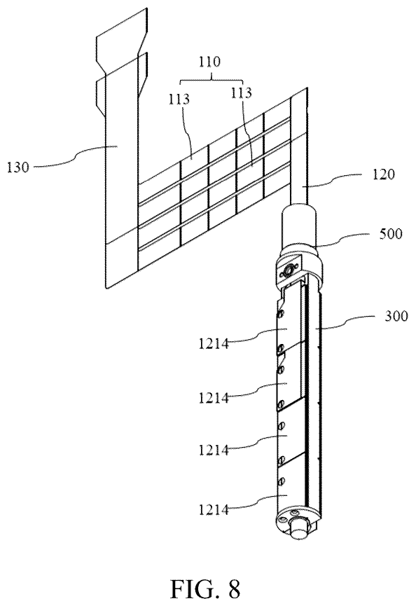

In some embodiments, referring to FIG. 2 and FIG. 8, each of the connecting units 121 includes a connecting section 1214; when the connecting units 121 are folded, the connecting sections 1214 of the connecting units 121 are staggered (i.e., misaligned) to be electrically connected to the transducer 300 respectively. By the staggered arrangement of the connecting sections 1214, the connecting sections 1214 are arranged in a staggered manner (e.g., not overlapped) after the connecting units 121 are folded, so that the connecting units 121 can be connected to the transducer 300.

Preferably, referring to FIG. 2 and FIG. 8, the connecting sections 1214 are sequentially staggered along the second direction Y. In some examples, projections of the connecting sections 1214 on a plane parallel to the direction Y are not overlapped. The second direction Y is perpendicular to the first direction X, and the second direction Y is parallel to an axial direction of the transducer 300 and an axial direction of the main shaft 200, so that the connecting sections 1214 in the connecting units 121 can be electrically connected to different positions of the transducer 300 along the axial direction in sequence. The transducer 300 includes a plurality of array elements distributed sequentially along the axial direction, and each connecting section 1214 is electrically connected to one or more array elements.

In some embodiments, referring to FIG. 1, FIG. 2 and FIG. 8, the connecting sections 1214 are provided with an alignment hole 1213 and gold fingers, and during assembly, the alignment holes 1213 of the connecting sections 1214 can be respectively positioned at different positions of the transducer 300 along the axial direction, and then, the gold fingers of the connecting sections 1214 are respectively soldered to FPCs on the array elements, so as to electrically connect the connecting units 121 to the transducer 300.

Optionally, one, two, or more alignment holes 1213 on each connecting section 1214 may be provided.

In some embodiments, referring to FIG. 2, each of the connecting units 121 further includes a folding section 1215, one end of the folding section 1215 is connected to the winding portion 110, and the other end of the folding section 1215 is connected to the connecting section 1214. The connecting section 1214 includes a convex portion 1216 extending outwards in the first direction X relative to the folding section 1215, and the alignment hole 1213 is formed in the convex portion 1216. A size of the convex portion 1216 in the first direction X is larger than that of the folding section 1215 in the first direction X. The alignment hole 1213 is configured for alignment with the transducer 300. The arrangement of the convex portion 1216 facilitates positioning of the alignment hole 1213 and the array element of the transducer 300, and the convex portion 1216 is cut away after the gold finger on the connecting section 1214 is soldered to the FPC of the array element.

In the present application, the winding portion 110 may include one winding unit 113 or at least two winding units 113 according to actual design requirements and assembly requirements. The winding unit 113 may also be understood as a winding member which is a windable part in the flexible circuit board 100. The winding portion 110 is windable in the first direction X.

As an example, referring to FIG. 4, the winding portion 110 includes one winding unit 113. Specifically, the winding unit 113 extends along the first direction X from the first end 111 to the second end 112, and each of the connecting units 121 is connected to the first end 111 of the winding unit 113.

As an example, referring to FIG. 1 to FIG. 3, the winding portion 110 includes at least two winding units 113 arranged in the second direction Y. For example, FIG. 3 shows four winding units 113. The number of the winding units 113 is the same as that of the connecting units 121, and the connecting units 121 are connected to the winding units 113 in a one-to-one correspondence manner. Specifically, the winding units 113 are sequentially distributed along the second direction Y, and the winding units 113 extend from the first end 111 to the second end 112 along the first direction X; the connecting units 121 are sequentially distributed along the first direction X, the connecting units 121 extend along the second direction Y from a fixed end 1212 to a connecting end 1211, and the winding units 113 are perpendicularly connected to the connecting units 121 in a one-to-one correspondence manner. In addition, lengths of the winding units 113 in the first direction X are different, and lengths of the connecting units 121 in the second direction Y are different, so that the winding units 113 arranged side by side in the second direction Y can be perpendicularly connected to the connecting units 121 arranged side by side in the first direction X in the one-to-one correspondence manner, and the connecting sections 1214 of the folded connecting units 121 can be staggered in the second direction Y to be respectively connected to different positions of the transducer 300 in the axial direction.

As an example, the winding portion 110 includes at least two winding units 113, the number of the winding units 113 is smaller than that of the connecting units 121, and each winding unit 113 is connected to at least one connecting unit 121. For example, if the number of the winding units 113 is two and the number of the connecting units 121 is four, every two connecting units 121 may be connected to one of the winding units 113; alternatively, if the number of the winding units 113 is two and the number of the connecting units 121 is three, one of the winding units 113 may be connected to two of the connecting units 121, and the other winding unit 113 may be connected to the remaining one of the connecting units 121.

As an example, the winding portion 110 includes at least two winding units 113, the number of the winding units 113 is larger than that of the connecting units 121, and each connecting unit 121 is connected to at least one winding unit 113. For example, if the number of the connecting units 121 is two and the number of the winding units 113 is four, every two winding units 113 may be connected to one of the connecting units 121.

In some embodiments, referring to FIG. 1 to FIG. 4, the length extension direction of the winding portion 110 and the length extension direction of the transduction connecting portion 120 are perpendicular to each other, for example, at least at their connecting position; the length extension direction of the winding portion 110 and the length extension direction of the adapter connecting portion 130 are perpendicular to each other, for example, at least at their connecting position.

As defined above, the length extension direction of the winding portion 110 is the extension direction from the first end 111 to the second end 112 of the winding portion 110. The length extension direction of the transduction connecting portion 120 is the extension direction from the fixed end 1212 to the connecting end 1211 of the transduction connecting portion 120, i.e., the second direction Y. The length extension direction of the adapter connecting portion 130 is a direction extending from an end thereof connected to the adapter board 400 to the winding portion 110. The second direction Y is generally arranged parallel to the axial direction of the main shaft 200 during assembly.

In this embodiment, the length extension direction of the winding portion 110 is perpendicular to the length extension direction of the transduction connecting portion 120, for example, at least at their connecting position, and the length extension direction of the winding portion 110 is perpendicular to the length extension direction of the adapter connecting portion 130, for example, at least at their connecting position, so that after the winding portion 110 is wound around the main shaft 200, the transduction connecting portion 120 can further extend along the axial direction of the main shaft 200 to be electrically connected to different positions of the transducer 300 along the axial direction, and the adapter connecting portion 130 can further extend along the axial direction of the main shaft 200 to be attached to an outer wall of a protection member 700, and extend out of a rear shell 800 to be electrically connected to the adapter board 400. The protection member 700 is a cylindrical structure sleeved over the main shaft 200 for protecting the flexible circuit board 100, and the rear shell 800 is sleeved over the protection member 700.

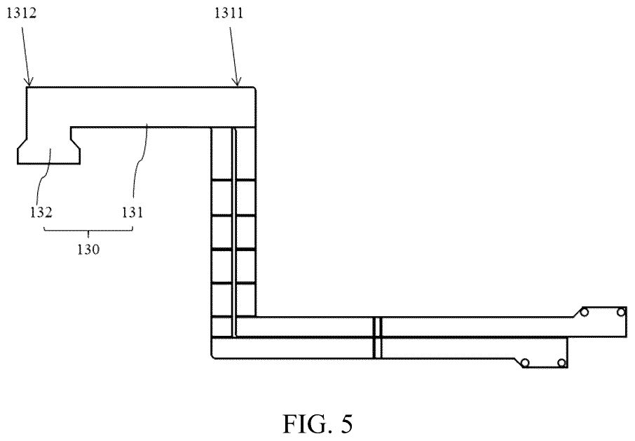

In some embodiments, referring to FIG. 1 and FIG. 5, the adapter connecting portion 130 includes a fixed section 131 and an inserting section 132; the fixed section 131 is connected to the second end 112 of the winding portion 110, the fixed section 131 is fixed relative to the adapter board 400, and specifically, the fixed section 131 is fixed to the protection member 700 of the ultrasonic probe; the inserting section 132 is configured to be electrically connected to the adapter board 400, and specifically inserted into an electrical connector on the adapter board 400. The inserting section 132 extends in a parallel or bent manner from the fixed section 131.

It should be noted that the parallel extension of the inserting section 132 from the fixed section 131 means that the inserting section 132 extends continuously along a length extension direction of the fixed section 131, and the length extension direction of the fixed section 131 is a direction in which the fixed section 131 extends from a third end 1311 connected to the winding portion 110 along a fourth end 1312 facing away from the winding portion 110, that is, the entire adapter connecting portion 130 extends linearly along the same direction. For example, FIG. 4 shows the inserting section extends in a parallel manner from the fixed section.

In addition, it should be noted that the bent extension of the inserting section 132 from the fixed section 131 means that the inserting section 132 extends continuously in a direction deviating from the length extension direction of the fixed section 131. Specifically, the inserting section 132 may extend perpendicularly relative to the fixed section 131, or may extend at an acute or obtuse angle relative to the fixed section 131. For example, FIG. shows the inserting section extends in a bent manner from the fixed section.

In this embodiment, the inserting section 132 extends in the parallel or bent manner from the fixed section 131, so that the inserting section 132 can be inserted according to an actual orientation of the adapter board 400, so as to ensure that the inserting section 132 can be inserted into the adapter board in parallel, and prevent the inserting section 132 from being bent or damaged due to inserting.

Optionally, the fixed section 131 is connected to the winding portion 110 perpendicularly, the fixed section 131 extends in a direction opposite to the second direction Y, and the inserting section 132 may extend in the direction opposite to the second direction Y or in the first direction X.

In a specific embodiment, the whole flexible circuit board 100 is roughly in a Z shape, the winding portion 110 extends linearly, the transduction connecting portion 120 extends linearly, the winding portion 110 extends linearly, and the length extension direction of the winding portion 110 is perpendicular to the length extension direction of the transduction connecting portion 120; the length extension direction of the winding portion 110 and the length extension direction of the adapter connecting portion 130 are perpendicular to each other.

In another aspect, referring to FIG. 6 and FIG. 7, an embodiment of the present application further provides an ultrasonic probe, including a main shaft 200, a transducer 300, an adapter board 400 and the above flexible circuit board 100; the transducer 300 being mounted at an output end of the main shaft 200; the flexible circuit board 100 including a winding portion 110, an adapter connecting portion 130 and a plurality of connecting units 121, the winding portion 110 being wound around a circumferential side wall of the main shaft 200, the folded connecting units 121 being respectively connected to the transducer 300, and the adapter connecting portion 130 being connected to the adapter board 400. In this embodiment, by the arrangement of the flexible circuit board 100, a space occupied by the folded connecting units 121 is small, so that each of the connecting units 121 can be conveniently connected to the transducer 300 through the main shaft 200, and an outer diameter of the main shaft 200 can be smaller.

In particular, the transducer 300 has a generally cylindrical shape. The transducer 300 includes a mounting base for fixing and supporting the entire transducer 300, a backing layer, a piezoelectric layer, an FPC, a matching layer, and an acoustically transparent layer, and the transducer 300 is connected to the output end of the main shaft 200 through the mounting base. The output end of the main shaft 200 is an end of the main shaft 200 axially adjacent to the transducer 300. The backing layer is configured to absorb sound waves generated by a piezoelectric ultrasonic transducer, so as to avoid image artifacts; the piezoelectric layer is configured to transmit and receive ultrasonic waves; the FPC is configured to be connected to electrodes of array elements of the piezoelectric layer;

the matching layer is configured to solve the problem of mismatching of acoustic impedance of the ultrasonic wave on an interface between human tissue and the piezoelectric layer, and improve a transmission efficiency of the ultrasonic wave; the acoustically transparent layer is configured to focus an acoustic beam of a catheter transducer in an elevation direction to enhance sensitivity of the transducer 300 and improve image quality.

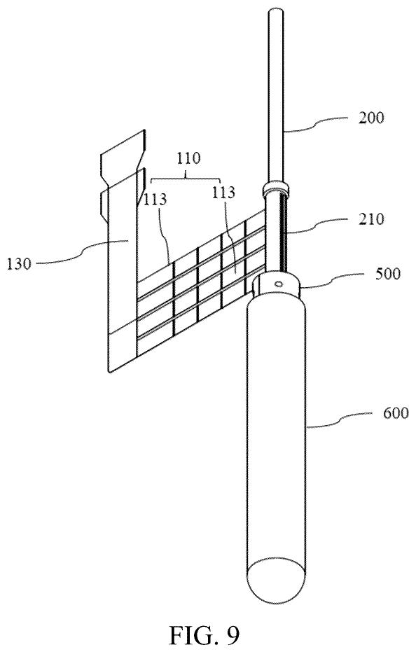

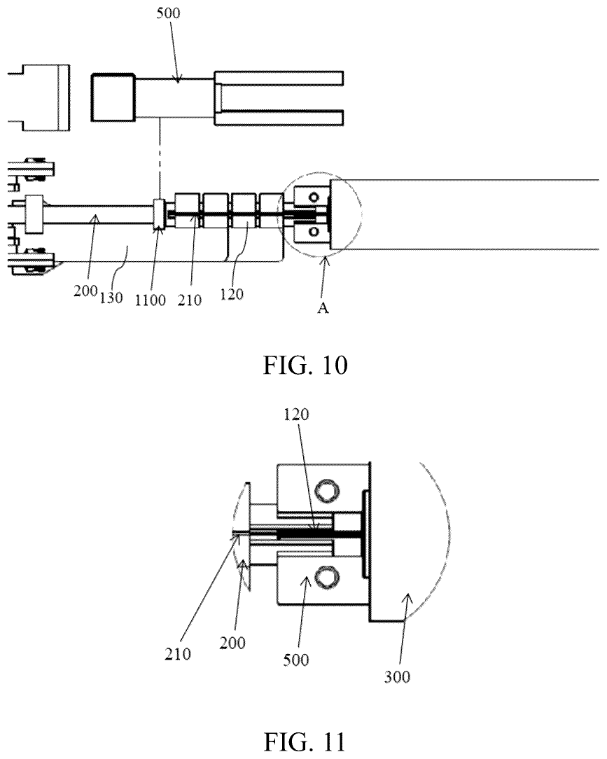

In some embodiments, referring to FIG. 9 to FIG. 11, the main shaft 200 is provided with a slot 210, the slot 210 extends along an axial direction of the main shaft 200, the slot 210 at least penetrates a circumferential side wall of the main shaft 200, and the slot 210 penetrates the output end of the main shaft 200; the folded connecting units 121 are inserted into the slot 210 and extend out from the output end of the main shaft 200 to be electrically connected to the transducer 300. Exemplarily, the folding sections 1215 of the folded connecting units 121 are inserted into the slot 210, and the connecting sections 1214 of the folded connecting units 121 extend out from the output end of the main shaft 200. In this embodiment, it is only necessary to provide the slot 210 in the main shaft 200 and enable a width of the slot 210 to match a total thickness of the plurality of folded connecting units 121, for example, enable the width of the slot 210 to be equal to or slightly greater than the total thickness of the plurality of folded connecting units 121. Since the flexible circuit board 100 is thin, the width of the slot 210 may be smaller, so that a diameter of the main shaft 200 may be designed to be smaller, the main shaft 200 may be connected to a motor through a coupler, and there is no need to form a connection between an input end of the main shaft 200 and the motor 1000 through a primary gear structure.

In some embodiments, the slot 210 may pass through the circumferential side of the main shaft 200, and the connecting unit 121 may be inserted into the slot 210 from the circumferential side of the main shaft 200 and extend out from the output end of the main shaft 200 to be electrically connected to the transducer 300 during assembly. In addition, the connecting unit 121 may be adhesively fixed in the slot 210 by adhesive dispensing.

In some other embodiments of the present application, the slot 210 may pass through two circumferentially opposite sides of the main shaft 200, and the connecting unit 121 may be inserted into the slot 210 from one of the two opposite sides of the main shaft 200. In addition, the connecting unit 121 may be adhesively fixed in the slot 210 by adhesive dispensing.

In some other embodiments of the present application, the slot 210 may penetrates the circumferential side wall of the main shaft 200, at least one insertion opening is formed on the circumferential side wall of the main shaft 200 opposite to the slot 210, at a position corresponding to the winding portion 110, and the winding portion 110 can pass through the insertion opening. During assembly, the folded connecting units 121 may be inserted into the slot 210 from the circumferential side of the main shaft 200, and the winding portion 110 may pass out from the circumferential side of the slot 210 and/or the insertion opening to be wound around the main shaft 200. In addition, the connecting unit 121 may be adhesively fixed in the slot 210 by adhesive dispensing.

Optionally, referring to FIG. 9, the slot 210 passes through a central axis of the main shaft 200, and a depth of the slot 210 along the first direction X can be relatively large under the condition that the diameter of the main shaft 200 is fixed. In other words, when the width of the folded connecting units 121 in the first direction X is constant, the diameter of the main shaft 200 can be designed to be smaller by designing the slot 210 to pass through the central axis of the main shaft 200. It is understood that, in other embodiments of the present application, the slot 210 may not pass through the central axis of the main shaft 200, which is not limited herein.

In some embodiments, referring to FIG. 6 to FIG. 11, the ultrasonic probe further includes a connector 500, the transducer 300 and the main shaft 200 being connected through the connector 500, and each connecting unit 121 being connected to the transducer 300 via the interior of the connector 500. By the arrangement of the connector 500, the connecting units 121 can extend out from the output end of the main shaft 200, are inserted into and connected to the transducer 300 via the interior of the connector 500, and thus are sealed inside the transducer 300.

Specifically, referring to FIG. 11, the connector 500 is cylindrical, the output end of the main shaft 200 is inserted into one end of the connector 500 and is connected to the connector 500, the transducer 300 is inserted into the other end of the connector 500 and is connected to the connector 500, and each of the connecting units 121 passes through a central hole of the connector 500.

Specifically, the connector 500 includes two semicircular structures which respectively sleeve over the output end of the main shaft 200 and an input end of the transducer 300 and are locked by screws, so as to form the connection between the main shaft 200 and the transducer 300.

In some embodiments, after each connecting unit 121 is connected to the transducer 300, the entire surface of the transducer 300 is encapsulated into a cylindrical shape by glue filling, so that the connecting position between the transducer 300 and the connecting unit 121 can be sealed. It is understood that, in other embodiments of the present application, the sealing of the transducer 300 may be formed by sleeving the surface of the transducer 300 with a sealing sleeve.

In some embodiments, the transducer 300 includes a plurality of array elements, each connecting unit 121 corresponds to one array element, or each connecting unit 121 corresponds to a plurality of array elements.

In some embodiments, referring to FIG. 7, the ultrasonic probe further includes a front shell 600, the front shell 600 is sleeved over the transducer 300 and defines a sealing cavity together with the connector 500, the sealing cavity is filled with coupling liquid, and the coupling liquid is isolated from the flexible circuit board 100. That is, the coupling liquid does not contact the flexible circuit board 100.

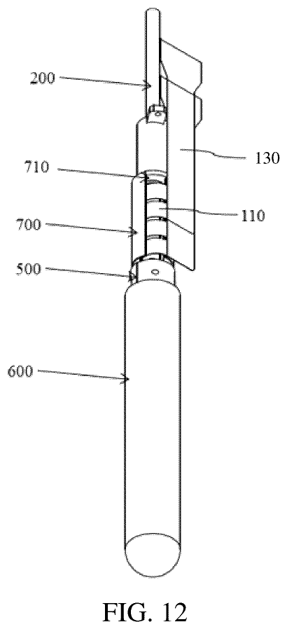

In some embodiments, referring to FIG. 6, FIG. 7 and FIG. 12, the ultrasonic probe further includes a protection member 700 fixed relative to the adapter board 400, the protection member 700 is substantially cylindrical, the protection member 700 is rotatably sleeved over the main shaft 200, the winding portion 110 is wound between the main shaft 200 and the protection member 700, and a window 710 for the adapter connecting portion 130 to pass through is formed on a circumferential side wall of the protection member 700. The arrangement of the protection member 700 can limit the winding portion 110 between the protection member 700 and the main shaft 200 to avoid that the winding portion 110 is excessively loosened due to rotation of the main shaft 200, and meanwhile can fix the adapter connecting portion 130, and specifically, the adapter connecting portion 130 is bonded to an outer wall of the protection member 700, so that connection reliability of the adapter connecting portion 130 and the adapter board 400 is ensured.

In some embodiments, referring to FIG. 6 and FIG. 7, the protection member 700 is rotatably sleeved over the main shaft 200 through a bearing 1100. Specifically, the protection member 700 is fixed in a handle of the ultrasonic probe and fixed on a same bracket as the motor 1000.

Optionally, the protection member 700 has two windows 710, the two windows 710 being oppositely arranged.

In the present application, the main shaft 200 and the protection member 700 rotate relatively, and the length of the winding portion 110 needs to be designed reasonably to ensure that the winding portion 110 is not damaged between the main shaft 200 and the protection member 700. The length of the winding portion 110 refers to a length from the first end 111 of the winding portion 110 to the second end 112 of the winding portion 110. In other words, the length of the winding portion 110 is defined as the distance between the two farthest ends of the winding portion 110 along the X direction.

Specifically, there are two winding modes for the winding portion 110, and the length of the winding portion 110 is calculated for each of the two winding modes. It should be noted that one or more flexible circuit boards 100 may be arranged in the ultrasonic probe, and when two flexible circuit boards 100 are arranged in the ultrasonic probe, the winding portions 110 of the two flexible circuit boards 100 may be wound in a staggered manner in a radial direction of the main shaft 200, or the two winding portions 110 may be wound in a staggered manner in the axial direction of the main shaft 200; when a plurality of flexible circuit boards 100 are arranged in the ultrasonic probe, the winding portions 110 of the respective flexible circuit boards 100 may be wound in a staggered manner in the radial direction of the main shaft 200 and/or in a staggered manner in the axial direction of the main shaft 200.

Since the winding mode and the length calculation of the winding portion 110 are the same under the above conditions, the condition where two flexible circuit boards 100 are arranged in the ultrasonic probe is described below. Specifically, the two transduction connecting portions 120 are respectively inserted into the slot 210 of the main shaft 200, and the two winding portions 110 are respectively wound around the main shaft 200 and respectively led out from the two oppositely arranged windows 710 of the protection member 700 to be attached to the outer wall of the protection member 700.

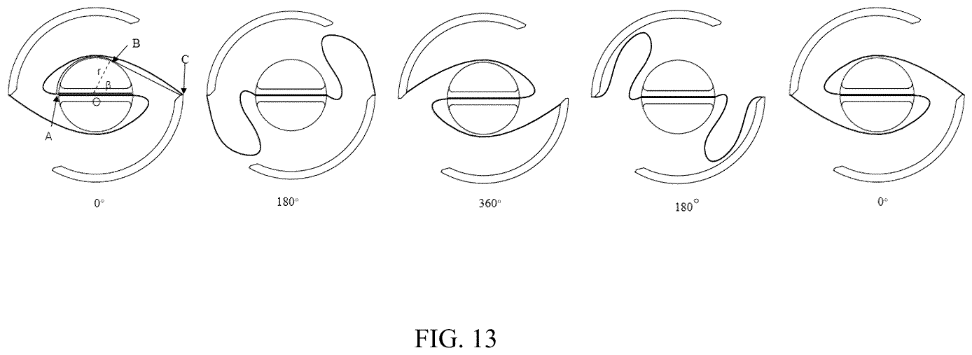

Under the first condition, referring to FIG. 13, a bending direction of the winding portion 110 changes during rotation, and specifically, a winding direction of the winding portion 110 changes during the process of rotating the main shaft 200 from 0° to 360°, for example, changes from clockwise winding to counterclockwise winding. In this case, the winding portion 110 is relatively short, but a bending angle is large each time the winding portion 110 is rotated. If the length of the winding portion 110 is designed and calculated according to a median value, the winding portion 110 can be ensured to be in a loose state all the time, and the bending angle is as small as possible.

Specifically, in an initial state, the two winding portions 110 are respectively led out from two opposite sides of the slot 210 of the main shaft 200, wound in a loose state for a half turn, and then attached to the outer side wall of the protection member 700, and the length of the winding portion 110 is L. As shown in FIG. 13, after the main shaft 200 rotates clockwise by 180° and 360° from an initial position, it rotates counterclockwise by 180° and 360° back to the initial position. There are two limit cases for the length L of the winding portion 110:
first limit case: referring to the initial position of FIG. 13 (i.e., the position shown at 0° in FIG. 13), the winding portion 110 reaches point B along an outer wall of the main shaft 200 from point A, and then linearly reaches point C, and in this case, the length is L₁;

$${L}_{1} = \frac{180 - β}{360} \cdot 2 \cdot π \cdot r + {L}_{BC} , wherein β = arccos ( \frac{r}{{R}_{1}} ) , {L}_{BC} = \sqrt{{{R}_{1}}^{2} - {r}^{2}} ,$$

r is the outer diameter of the main shaft 200, R₁ is an inner diameter of the protection member 700, and L_{BC} is a distance from the point B to the point C. It should be noted that the arc AB is not necessarily tightly attached to the main shaft 200, so that the value of L₁ may be scaled up to some extent, for example, by 10% to 30%, when L₁ is calculated.

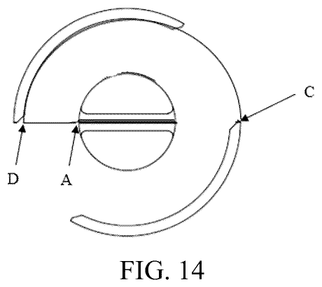

Second limit case: referring to FIG. 14, the winding portion 110 reaches point D from the point A and reaches the point C along an inner wall of the protection member 700, and in this case, the length is L₂;

$${L}_{2} = \frac{180}{360} \cdot 2 \cdot π \cdot {R}_{2} + {L}_{AD} ;$$

wherein L_{AD}=R₂−r; R₂ is the outer diameter of the protection member 700.

As shown, L₁≤L≤L₂, and when the length L is closer to L₁, the design is more reasonable.

Under the second condition, the bending direction of the winding portion 110 does not change during the rotation, and specifically, the winding direction of the winding portion 110 does not change during the rotation of the main shaft 200, for example, the winding portion is wound clockwise or counterclockwise all the time. In this case, the bending angle of the winding portion 110 is small, but a number of winding turns of the winding portion 110 is large, and in this case, designing and calculation of the winding portion 110 are performed according to a minimum length.

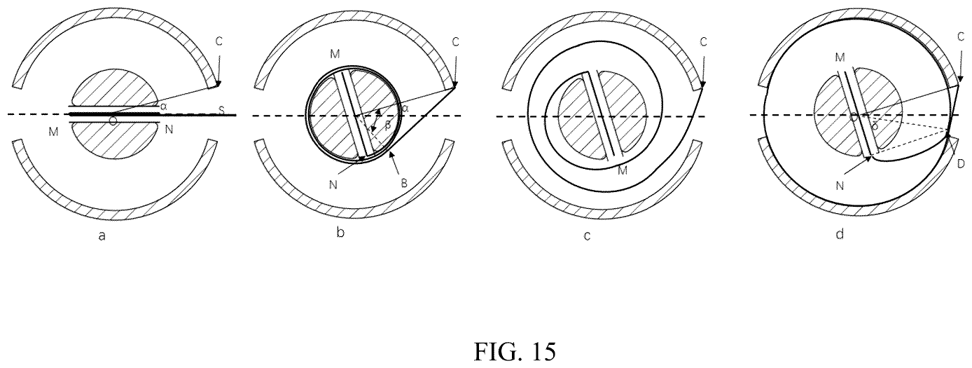

Referring to FIG. 15, a in FIG. 15 is an initial assembly state in which opposite openings of the slot 210 of the main shaft 200 are aligned with the two windows 710 of the protection member 700; b in FIG. 15 is a state where the protection member 700 is fixed, and the main shaft 200 rotates clockwise by γ degrees, wherein γ is greater than 360, and in this case, the winding portion 110 is fully wound and tightly attached to the main shaft 200, and the winding portion 110 is at a first extreme position; c in FIG. 15 is a state where the protection member 700 is fixed, and the main shaft 200 is rotated counterclockwise by 180° relative to b in FIG. 15; d in FIG. 15 is a state where the protection member 700 is fixed, and the main shaft 200 is rotated counterclockwise by 360° relative to b in FIG. 15, and in this case, the winding portion 110 is fully unwound and attached to the inner wall of the protection member 700, and the winding portion 110 is at a second extreme position. In the drawing, M and N are positions of the openings in the two sides of the main shaft 200 respectively, and the winding initial position of the winding portion 110 is always the point N; the point C is an initial position where the winding portion 110 is fixed to the protection member 700; O is the circle center; D is the intersection of a vertical line ND of MN at the maximum winding radius.

As shown in b of FIG. 15, when the winding portion 110 is at the first extreme position, the main shaft 200 is rotated by γ, the minimum length of the winding portion 110 is L₁, and

$${L}_{1} = \frac{γ + α - β}{360} \cdot 2 \cdot π \cdot r + {L}_{BC} ,$$

wherein

$$β = arccos ( \frac{r}{{R}_{1}} ) , and {L}_{BC} = \sqrt{{{R}_{1}}^{2} - {r}^{2}} ;$$

r is the outer diameter of the main shaft 200; R₁ is the inner diameter of the protection member 700; γ is a maximum rotation angle of the main shaft 200 during working; α is the angle of the single side window 710 of the protection member 700, i.e., the angle ∠COS between the connecting line OC and the center line of the opening of the main shaft 200, and in order to ensure that the arc length of the protection member 700 is enough to support the winding portion 110, 0°≤α≤45°.

As shown in d of FIG. 15, when the winding portion 110 is at the second extreme position, the main shaft 200 is rotated by (γ−360) degrees, the maximum length of the winding portion 110 is L₂, and

$${L}_{2} = \frac{γ + α - 360 - δ}{360} \cdot 2 \cdot π \cdot P + {S}_{ND} ,$$

wherein

$$δ = arccos ( \frac{r}{{R}_{2}} ) ;$$

P is the winding radius, and due to the limitation of the size of the protection member 700, r<P≤R₂, and R₂ is the outer diameter of the protection member 700; S_{ND} is the ND arc length, wherein when S_{ND} satisfies the following relationship,

$${L}_{ND} < {S}_{ND} < {R}_{2} - r + \frac{δ}{360} \cdot 2 \cdot π \cdot {R}_{2} .$$

A minimum value of L needs to be solved, and by the following relationships: L=L₁ and L≤L₂, the following formula is deduced:

$${L}_{min} = \frac{γ + α - β}{360} \cdot 2 \cdot π \cdot r + {L}_{BC} ; and$$

$$\frac{γ + α - β}{360} \cdot 2 \cdot π \cdot r + {L}_{BC} \leq \frac{γ + α - 360 - δ}{360} \cdot 2 \cdot π \cdot P + {S}_{ND} .$$

In the above formula, in order to simplify calculation, S_{ND} can be evaluated according to L_{BC}, and therefore, the following formula is derived from the above formula:

$$γ + α \geq \frac{1032.4}{P - 2.9} + 420.$$

It can be seen that the larger P, the smaller γ+α; the smaller γ+α, the smaller L.

Therefore, when P=R₂, (γ+α)ₘᵢₙ can be calculated, and thus, Lₘᵢₙ can be calculated.

Generally, both the first and second extreme positions have reserved margins θ, and therefore,

$${L}_{1} = \frac{γ + α - β + θ}{360} \cdot 2 \cdot π \cdot r + {L}_{BC}$$

$${L}_{2} = \frac{γ + α - 360 - δ - θ}{360} \cdot 2 \cdot π \cdot P + {S}_{ND}$$

wherein θ is typically 10° to 20°, and Lₘᵢₙ is also calculated in the manner described above.

In the present application, in order to make the length of the winding portion 110 smaller during winding, the main shaft 200 and the protection member 700 may be further improved as follows based on the above calculation method.

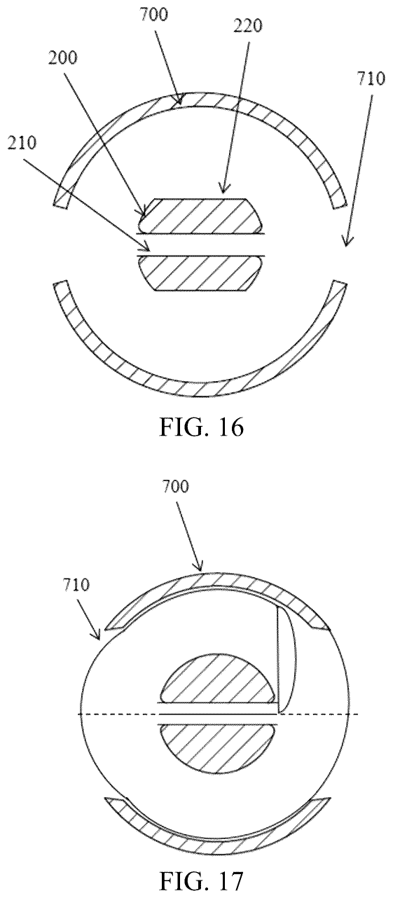

Referring to FIG. 16, for the main shaft 200, a notch 220 is formed in the circumferential side wall of the main shaft 200, and specifically, the notch 220 may be formed by removing materials from the circumferential side wall of the main shaft 200 along the first direction, and the first direction is a direction perpendicular to the slot 210. The arrangement of the notch 220 can reduce an outer circumference of the main shaft 200, and make an equivalent winding radius rs of the winding portion 110 smaller, so that Lₘᵢₙ is smaller.

For the protection member 700, referring to FIG. 17, the two windows 710 of the protection member 700 are designed to have larger sizes, and the central angle corresponding to the window 710 is ensured to be smaller than 90 degrees, that is, a is ensured to be smaller than 45 degrees, and in this case, the equivalent winding radius R₂ₛ can be made larger, so that Lₘᵢₙ is smaller. Here, the central angle corresponding to the window 710 refers to the included angle between the lines connecting the two ends of the window 710 to the central axis of the main shaft 200.

In general, the above two modes can make the winding angle of the winding portion 110 smaller, the winding length of the winding portion 110 smaller, and winding reliability higher.

In some embodiments, referring to FIG. 7, the ultrasonic probe further includes a rear shell 800 and the handle, the adapter board 400 is fixed in the handle, the rear shell 800 is sleeved over the protection member 700, the rear shell 800 is connected between the handle and the front shell 600, the adapter connecting portion 130 passes between the rear shell 800 and the protection member 700 to be connected to the adapter board 400, the fixed section 131 of the adapter connecting portion 130 is pasted to the outer wall of the protection member 700, and the adapter connecting portion 130 is fixed by the protection member 700, so that connection reliability of the adapter connecting portion 130 and the adapter board 400 is ensured.

Optionally, the ultrasonic probe further includes a motor 1000, the motor 1000 is mounted in the handle, and the motor 1000 is connected to the input end of the main shaft 200.

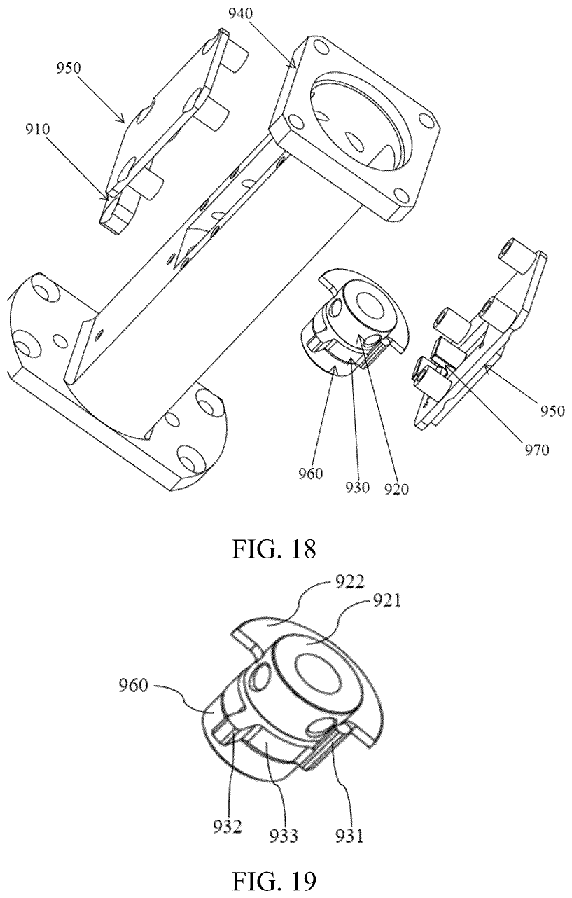

In some embodiments, referring to FIG. 6, FIG. 7, and FIG. 18 to FIG. 20, the ultrasonic probe further includes a limiting assembly 900, the limiting assembly 900 being configured to limit a rotating stroke of the main shaft 200. In the present application, since a three-dimensional image needs to be acquired, the main shaft 200 is required to drive the transducer 300 to rotate by 360 degrees. Meanwhile, since the adapter connecting portion 130 is connected to the adapter board 400, and the transduction connecting portion 120 is connected to the transducer 300, that is, the two ends of the winding portion 110 between the adapter connecting portion 130 and the transduction connecting portion 120 rotate relatively during the rotation of the main shaft 200, in order to avoid damage to the flexible circuit board 100 caused by the rotation of the main shaft 200, the rotating stroke of the main shaft 200 needs to be limited by the limiting assembly 900, so as to reduce the damage to the flexible circuit board 100.

In some embodiments, referring to FIG. 18 to FIG. 20, the limiting assembly 900 includes a blocking structure 910, a first limiting member 920 and a second limiting member 930. The blocking structure 910 is fixed relative to the adapter board 400, and the blocking structure 910 includes a first blocking portion 911 and a second blocking portion 912 arranged at intervals along a circumferential direction; the first limiting member 920 is fixedly connected to the main shaft 200, the first limiting member 920 includes an arc-shaped portion 922 surrounding the main shaft 200, and the arc-shaped portion 922 has a first limiting surface 9221 and a second limiting surface 9222 which are arranged oppositely along the circumferential direction; the second limiting member 930 is rotatably sleeved over the main shaft 200, the second limiting member 930 includes a first limiting portion 931 and a second limiting portion 932, and the first limiting portion 931 is movably arranged between the first limiting surface 9221 and the second limiting surface 9222; the second limiting portion 932 is movably arranged between the first blocking portion 911 and the second blocking portion 912.

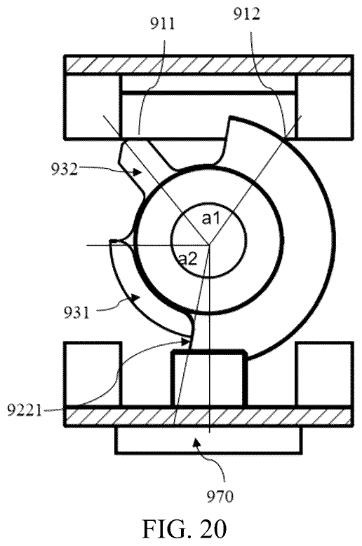

The blocking structure 910 is fixed relative to the adapter board 400, that is, the blocking structure 910 is fixedly arranged during the rotation of the main shaft 200. The first limiting member 920 is fixedly connected to the main shaft 200, the first limiting member 920 rotates along with the main shaft 200, and the arc-shaped portion 922 also rotates along with the main shaft 200. The second limiting member 930 is rotatably sleeved over the main shaft 200, so that the second limiting member 930 and the main shaft 200 can rotate relatively. Specifically, as shown in FIG. 20, the first limiting member 920 and the second limiting member 930 are in a first state, and in this case, the second limiting portion 932 is limited at the first blocking portion 911 of the blocking structure 910, and the first limiting portion 931 abuts against the first limiting surface 9221 of the arc-shaped portion 922, so that the first limiting member 920 and the second limiting member 930 cannot rotate clockwise. In this case, the first limiting member 920 can be driven to rotate counterclockwise, and after the first limiting member 920 rotates counterclockwise by an angle A1, the first limiting portion 931 abuts against the second limiting surface 9222 of the first limiting member 920, the second limiting surface 9222 pushes the second limiting member 930 and the first limiting member 920 to continue to rotate counterclockwise together, and after the second limiting member 930 and the first limiting member 920 rotate counterclockwise by an angle A2 together, the second limiting portion 932 of the second limiting member 930 abuts against the second blocking portion 912 of the blocking structure 910, so that the first limiting member 920 and the second limiting member 930 cannot continue to rotate clockwise, that is, the first limiting member 920 and the second limiting member 930 can only rotate clockwise in this case. A total angle A0=A1+A2 that the main shaft 200 can rotate counterclockwise is only required to satisfy the condition that A1+A2 is greater than 360 and less than 450.

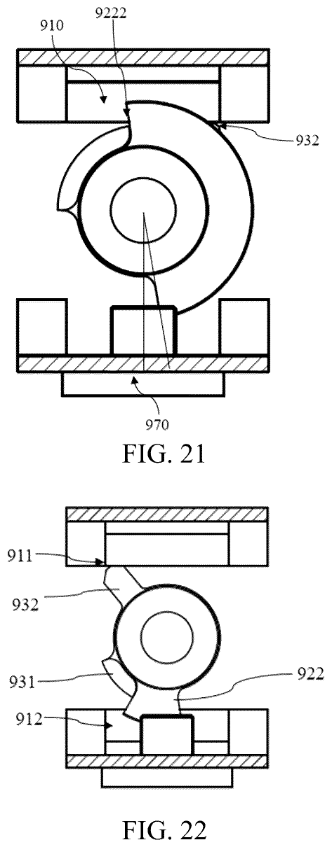

As an example, referring to FIG. 18 and FIG. 21, if an included angle between the first limiting surface 9221 and the second limiting surface 9222 is 180°, and an included angle a2 corresponding to the first limiting portion 931 along the circumferential direction is 80°, A1=180°−80°=100°; further, the central angle a1 corresponding to the first blocking portion 911 and the second blocking portion 912 of the blocking structure 910 along the circumferential direction is 80°, that is, the angle by which the second limiting portion 932 can be rotated from the first blocking portion 911 to the second blocking portion 912 of the blocking structure 910 is A2=360°−80°=280°, and therefore, A0=100°+280°=380°, which can ensure complete circular motion scan, and the rotation angle of the main shaft 200 is −10° to 370° as indicated by the two extreme positions in FIG. 20 and FIG. 21, which satisfies design requirements.

Specifically, in this example, the first blocking portion 911 and the second blocking portion 912 are integrally connected, and therefore, the blocking structure 910 is a block-shaped structure extending along the circumferential direction of the main shaft 200.

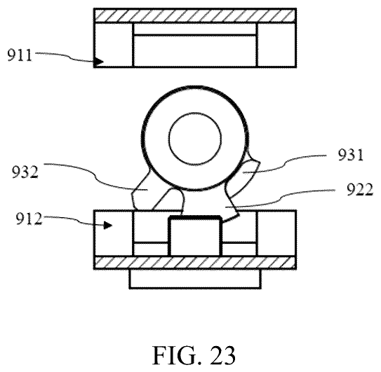

As another example, referring to FIG. 22 and FIG. 23, the rotation angle A1 of the first limiting portion 931 between the first limiting surface 9221 and the second limiting surface 9222 is 280°; in addition, since the circumferential angle between the first blocking potion 911 and the second blocking potion 912 is 100°, that is, A2 is 100°, A0=100°+280°=380°, which can ensure complete circular motion scan to satisfy the design requirements.

Specifically, in this embodiment, the first blocking portion 911 and the second blocking portion 912 are independently arranged and spaced apart in the circumferential direction of the main shaft 200.

In some embodiments, referring to FIG. 18, the limiting assembly 900 further includes a fixed bracket 940 and two mounting plates 950, the two mounting plates 950 are respectively mounted on two opposite sides of the fixed bracket 940, the fixed bracket 940 is fixedly connected to the handle, the blocking structure 910 is mounted on the fixed bracket 940, the main shaft 200 is arranged through the fixed bracket 940, the first limiting member 920 is fixedly sleeved over the main shaft 200 and rotates between the two mounting plates 950, the second limiting member 930 is rotatably sleeved over the main shaft 200, the main shaft 200 is sleeved with a stop ring 960, and the second limiting member 930 is axially limited by the stop ring 960, so as to prevent the second limiting member 930 from being separated from the main shaft 200.

Specifically, referring to FIG. 19, the first limiting member 920 includes a sleeving portion 921 and an arc-shaped portion 922 formed on a periphery of the sleeving portion 921, and the sleeving portion 921 is fixedly sleeved over the main shaft 200.

Specifically, referring to FIG. 19, the second limiting member 930 includes a rotating portion 933, the rotating portion 933 is rotatably sleeved over the main shaft 200, and the first limiting portion 931 and the second limiting portion 932 are respectively formed at different positions of the rotating portion 933 along the circumferential direction.

In some embodiments, referring to FIG. 18 and FIG. 20, the limiting assembly 900 further includes a photoelectric sensor 970, the photoelectric sensor 970 is arranged on a periphery of the arc-shaped portion 922, the photoelectric sensor 970 is fixed on the mounting plate 950, and when the arc-shaped portion 922 rotates through the photoelectric sensor 970, confirmation of the initial position is completed, which represents that ultrasonic scanning may be started.

Optionally, the blocking structure 910 and the photoelectric sensor 970 are mounted on the two mounting plates 950 respectively.

The flexible circuit board and the ultrasonic probe according to the application have the following beneficial effects: the transduction connecting portion is divided into the at least two connecting units, the at least two connecting units are foldable in the first direction, one end of each connecting unit is connected to the first end of the winding portion, and the other end of each connecting unit is configured to be electrically connected to the transducer, so that the electrical connection of the adapter board with the transducer can be realized, and meanwhile, the connecting units can be folded in the first direction during assembly, the total width of the folded connecting units in the first direction is equal to the width of one connecting unit, the width of the transduction connecting portion in the first direction is greatly reduced, and therefore, the transduction connecting portion occupies a small space during assembly and can be easily electrically connected to the transducer via the main shaft.

The above description is only a preferred embodiment of this application and is not intended to limit this application, and any modifications, equivalents and improvements made within the spirit and principle of the present application should be included in the protection scope of the present application.

What is claimed is:

1. A flexible circuit board for electrically connecting a transducer and a signal processing circuit in an ultrasonic probe, the flexible circuit board comprising:
a winding portion configured to be wound around a main shaft of the ultrasonic probe;
a transduction connecting portion comprising at least two connecting units arranged along a first direction, the at least two connecting units being capable of being folded along the first direction, one end of each connecting unit being connected to a first end of the winding portion, and the other end of each connecting unit being configured to be electrically connected to the transducer; and
an adapter connecting portion, one end of the adapter connecting portion being connected to a second end of the winding portion, and the other end of the adapter connecting portion being configured to be electrically connected to the signal processing circuit.

2. The flexible circuit board according to claim 1, wherein each of the connecting units comprise a connecting section, and when the connecting units are folded, the connecting sections are arranged in a staggered manner to be electrically connected to the transducer respectively.

3. The flexible circuit board according to claim 1, wherein the winding portion comprises one winding unit, and the connecting units are respectively connected to the winding unit; or
the winding portion comprises at least two winding units, a number of the winding units is the same as that of the connecting units, and the connecting units are connected to the winding units in a one-to-one correspondence manner; or
the winding portion comprises at least two winding units, the number of the winding units is smaller than that of the connecting units, and each winding unit is connected to at least one connecting unit; or
the winding portion comprises at least two winding units, the number of the winding units is larger than that of the connecting units, and each connecting unit is connected to at least one winding unit.

4. The flexible circuit board according to claim 1, wherein a length extension direction of the winding portion is perpendicular to a length extension direction of the transduction connecting portion.

5. The flexible circuit board according to claim 1, wherein the length extension direction of the winding portion is perpendicular to a length extension direction of the adapter connecting portion.

6. The flexible circuit board according to claim 1, wherein the adapter connecting portion comprises:
a fixed section, the fixed section being connected to the winding portion, and the fixed section being fixed relative to the signal processing circuit; and
an inserting section configured to be electrically connected to the signal processing circuit;
wherein the inserting section extends in a parallel or bent manner from the fixed section.

7. The flexible circuit board according to claim 1, wherein after the at least two connecting units are folded, a width of the transduction connecting portion in the first direction is less than a sum of widths of all the connecting units in the first direction.

8. The flexible circuit board according to claim 1, wherein after the at least two connecting units are folded, a width of the transduction connecting portion in the first direction is equal to a width of one connecting unit in the first direction.

9. The flexible circuit board according to claim 2, wherein each of the connecting units further comprises a folding section, one end of the folding section is connected to the winding portion, and the other end of the folding section is connected to the connecting section; and
the connecting section comprises a convex portion which extends outwards along the first direction relative to the folding section, and the convex portion is provided with an alignment hole for alignment with the transducer.

10. An ultrasonic probe, comprising:
a main shaft,
a transducer mounted at an output end of the main shaft,
a signal processing circuit and
a flexible circuit board for electrically connecting the transducer and the signal processing circuit, the flexible circuit board comprising:
a winding portion configured to be wound around a main shaft of the ultrasonic probe;
a transduction connecting portion comprising at least two connecting units arranged along a first direction, the at least two connecting units being capable of being folded along the first direction, one end of each connecting unit being connected to a first end of the winding portion, and the other end of each connecting unit being configured to be electrically connected to the transducer; and
an adapter connecting portion, one end of the adapter connecting portion being connected to a second end of the winding portion, and the other end of the adapter connecting portion being configured to be electrically connected to the signal processing circuit.

11. The ultrasonic probe according to claim 10, wherein the main shaft is provided with a slot, the slot at least penetrates a circumferential side wall of the main shaft, and the slot penetrates the output end of the main shaft; the folded connecting units are inserted into the slot and extend out of the output end of the main shaft to be electrically connected to the transducer.

12. The ultrasonic probe according to claim 10, wherein the transducer comprises a plurality of array elements distributed sequentially along an axial direction of the transducer, and each connecting section is electrically connected to one or more array elements.

13. The ultrasonic probe according to claim 10, wherein the ultrasonic probe further comprises a connector, the transducer and the main shaft being connected through the connector, and each connecting unit being connected to the transducer via the interior of the connector.

14. The ultrasonic probe according to claim 10, wherein the ultrasonic probe further comprises a protection member fixed relative to the signal processing circuit, the protection member being rotatably sleeved over the main shaft, the winding portion being wound between the main shaft and the protection member, and a circumferential side wall of the protection member being provided with a window for the adapter connecting portion to pass through.

15. The ultrasonic probe according to claim 14, wherein the adapter connecting portion is bonded to an outer wall of the protection member.

16. The ultrasonic probe according to claim 11, wherein at least one insertion opening is formed on the circumferential side wall of the main shaft opposite to the slot, at a position corresponding to the winding portion.

17. The ultrasonic probe according to claim 13, wherein the ultrasonic probe further comprises a limiting assembly, the limiting assembly being configured to limit a rotating stroke of the main shaft.

18. The ultrasonic probe according to claim 17, wherein the limiting assembly comprises:
a blocking structure comprising a first blocking portion and a second blocking portion spaced apart along a circumferential direction;
a first limiting member fixedly connected to the main shaft, the first limiting member comprising an arc-shaped portion surrounding the main shaft, and the arc-shaped portion having a first limiting surface and a second limiting surface which are oppositely arranged along the circumferential direction; and
a second limiting member rotatably sleeved over the main shaft, the second limiting member comprising a first limiting portion and a second limiting portion, and the first limiting portion being movably arranged between the first limiting surface and the second limiting surface; the second limiting portion being movably arranged between the first blocking portion and the second blocking portion.

19. The ultrasonic probe according to claim 13, wherein the circumferential side wall of the main shaft is provided with a notch for reducing an outer circumference of the main shaft.

20. The ultrasonic probe according to claim 14, wherein a length of the winding portion is determined according to an outer diameter of the main shaft, an outer diameter of the protection member, and an inner diameter of the protection member.
